(19) United States
(12) Patent Application Publication
(10) Pub. No.: US 2013/0209415 A1
(43) Pub. Date: Aug. 15, 2013

(54) PURIFIED COMPOSITIONS OF CARDIOVASCULAR PROGENITOR CELLS

(71) Applicants: Reza Ardehali, Redwood City, CA (US); Irving L. Weissman, Stanford, CA (US); Micha Drukker, Stanford, CA (US); Roeland Nusse, Stanford, CA (US)

(72) Inventors: Reza Ardehali, Redwood City, CA (US); Irving L. Weissman, Stanford, CA (US); Micha Drukker, Stanford, CA (US); Roeland Nusse, Stanford, CA (US)

(73) Assignee: The Board of Trustees of the Leland Stanford Junior University, Palo Alto, CA (US)

(21) Appl. No.: 13/701,425

(22) PCT Filed: Jun. 1, 2011
(86) PCT No.: PCT/US2011/038755

§ 371 Date: Apr. 26, 2013

Related U.S. Application Data
(60) Provisional application No. 61/396,943, filed on Jun. 3, 2010.

(51) Int. Cl. C12N 5/071

(52) U.S. Cl. CPC C12N 5/0657
USPC 424/93.7; 435/366

(57) ABSTRACT

Composition and methods are provided for the prospective enrichment of human cardiovascular progenitor cells, which can be differentiated into cardiomyocytes, from in vitro cultures of stem cells. The stem cells are cultured in conditions permissive for differentiation into cardiovascular progenitor cells, and cardiovascular progenitor cells are sorted for expression of one or more of the markers ROR2, CD13, KDR and PDGFαR, where the progenitor cells positively express these markers. Highly enriched populations of cardiomyocyte lineage cells can be obtained.

### BACKGROUND OF THE INVENTION

Regenerative medicine is the process of creating living, functional tissues to repair or replace tissue or organ function lost due to age, disease, damage, or congenital defects. This field holds the promise of regenerating damaged tissues and organs in the body by introducing outside cells, tissue, or even whole organs to integrate and become a part of tissues or replace whole organ. Importantly, regenerative medicine has the potential to solve the problem of the shortage of organs available for donation compared to the number of patients that require life-saving organ transplantation.

One key to the success of regenerative medicine strategies has been the ability to isolate and generate stem cells, including pluripotent stem cells. In one aspect, pluripotent stem cells can be differentiated into a necessary cell type, where the mature cells are used to replace tissue that is damaged by disease or injury. This type of treatment could be used to replace neurons damaged by spinal cord injury, stroke, Alzheimer's disease, Parkinson's disease, or other neurological problems. Cells grown to produce insulin could treat people with diabetes and heart muscle cells could repair damage after a heart attack. This list could conceivably include any tissue that is injured or diseased.

The generation of pluripotent stem cells that are genetically identical to an individual provides unique opportunities for basic research and for potential immunologically-compatible novel cell-based therapies. Methods to reprogram primate somatic cells to a pluripotent state include differentiated somatic cell nuclear transfer, differentiated somatic cell fusion with pluripotent stem cells, and direct reprogramming to produce induced pluripotent stem cells (iPS cells) (Takahashi K, et al. (2007) Cell 131:861-872; Park I H, et al. (2008) Nature 451:141-146; Yu J, et al. (2007) Science 318:1917-1920; Kim D, et al. (2009) Cell Stem Cell 4:472-476; Soldner F, et al. (2009) Cell. 136:964-977; Huangfu D, et al. (2008) Nature Biotechnology 26:1269-1275; Li W, et al. (2009) Cell Stem Cell 4:16-19).

A significant first hurdle in stem cell-based therapy is the differentiation of pluripotent cells into a desired tissue type. Such methods currently rely on the step-wise introduction of factors and conditions to guide the cells down a developmental pathway, resulting eventually in a mature or committed progenitor cell that can transplanted into a patient.

Muscle is one of the largest tissues in the body, and one that can be subjected to severe mechanical and biological stresses. A number of widespread and serious conditions cause necrosis of heart tissue, leading to unrepaired or poorly repaired damage. For example, coronary artery disease, in which the arteries feeding the heart narrow over time, can cause myocardial ischemia, which if allowed to persist, leads to heart muscle death. Another cause of ischemia is myocardial infarction (MI), which occurs when an artery feeding the heart suddenly becomes blocked. This leads to acute ischemia, which again leads to myocardial cell death, or necrosis.

Cardiac tissue death can lead to other heart dysfunctions. If the pumping ability of the heart is reduced, then the heart may remodel to compensate; this remodeling can lead to a degenerative state known as heart failure. Heart failure can also be precipitated by other factors, including valvular heart disease and cardiomyopathy. In certain cases, heart transplantation must be used to repair an ailing heart.

Unlike skeletal muscle, which regenerates from reserve myoblasts called satellite cells, the mammalian heart has a very limited regenerative capacity and, hence, heals by scar formation. The severity and prevalence of these heart diseases has led to great interest in the development of progenitor and stem cell therapy, which could allow the heart to regenerate damaged tissue and ameliorate cardiac injury (see Murry et al. (2002) C.S.H. Symp. Quant. Biol. 67:519-526). For human therapeutic application, a suitable myogenic cell type from either an autologous or appropriately matched allogeneic source may be delivered to the infarcted zone to repopulate the lost myocardium.

A number of different cell types have been considered for such therapies. While some researchers have reported the persistence of markers from somatic cells as diverse as hematopoietic stem cells; mesenchymal stem cells; and even peripheral blood cells; the evidence is, at least thus far, hotly disputed. While improvements can be found in some functional parameters, it does not seem that new myocytes are being produced.

Human ESC-derived cardiomyocytes possess the cellular elements required for electromechanical coupling with the host myocardium, such as gap and adherens junctions, and it is therefore expected that, when transplanted, these cells could electrically integrate and contribute to systolic function (see Mummery et al. (2003) Circulation 107:2733-2740). This property represents a significant advantage over other cell types, such as skeletal muscle, which act through modulation of diastolic function (see Reinecke et al. (2000) J. Cell. Biol. 149:731-740; and Reinecke et al. (2002) J. Mol. Cell. Cardiol. 34:241-249).

The clinical application of human embryonic stem cell (hESC)-derived products is limited by technical challenges, including the difficulty to isolate tissue-specific progenitors capable of tissue engraftment and regeneration. While several studies have reported efficient differentiation of hESCs towards cardiovascular lineages, the two most significant barriers to therapy remain the impurity of the final product and the unknown fate of these cells upon transplantation. The current “gold standard” method to evaluate the in vivo developmental potential and functional properties of cardiovascular progenitor cells is to transplant them into animal hearts (normally murine or porcine models). While the transplanted cells engraft into these heart models, it is unclear whether they functionally integrate. Hence, the developmental fates adopted by the hESC-derived cells cannot be elucidated with the current xenograft transplantation models, which is a necessary step prior to their use in regenerative therapy. Furthermore, the capacity of hESC-derived cardiovascular cells to functionally integrate into human tissues remains untested and unknown.

A system to prospectively isolate cardiovascular stem cells/progenitors and to evaluate their in vivo developmental potential in functioning human hearts will be an important step in clinical translation for myocardial regeneration.

### SUMMARY OF THE INVENTION

Composition and methods are provided for the prospective enrichment of human cardiovascular progenitor cells, which can be differentiated into cardiomyocytes, from in vitro cultures of stem cells. The stem cells are cultured in conditions permissive for differentiation into cardiovascular progenitor cells, and cardiovascular progenitor cells are sorted for expression of one or more of the markers ROR2, CD13, KDR and PDGFαR, where the progenitor cells positively express these markers. Highly enriched populations of cardiomyocyte lineage cells can be obtained.

The sorted cells are useful in transplantation, for experimental evaluation, and as a source of lineage and cell specific products, including mRNA species useful in identifying genes specifically expressed in these cells, and as targets for the discovery of factors or molecules that can affect them. Sorted cells may be used, for example, in a method of screening a compound for an effect on the differentiating cells of interest. This involves combining the compound with the cell population of the invention, and then determining any modulatory effect resulting from the compound. This may include examination of the cells for toxicity, metabolic change, or an effect on cell function.

In one embodiment of the invention, a population of cells is provided wherein the cells are substantially comprised of cells in the cardiomyocyte lineage. The cardiomyocyte lineage cells may be cardiomyocyte precursor cells, or differentiated cardiomyocytes. Differentiated cardiomyocytes include one or more of primary cardiomyocytes, nodal (pacemaker) cardiomyocytes; conduction cardiomyocytes; and working (contractile) cardiomyocytes, which may be of atrial or ventricular type. A medicament or delivery device containing cells of the invention is provided for treatment of a human or animal body, including formulations for cardiac therapy. Cardiomyocyte lineage cells may be administered to a patient in a method for reconstituting or supplementing contractile and/or pacemaking activity in cardiac tissue.

These and other embodiments of the invention will be apparent from the description that follows. The compositions, methods, and techniques described in this disclosure hold considerable promise for use in diagnostic, drug screening, and therapeutic applications.

### BRIEF DESCRIPTION OF THE FIGURES

FIG. 1. Identification of a cardiac mesoderm population marked by four surface markers: ROR2, CD13, KDR, and PDGFRα. a, Flow cytometric analysis of embryoid bodies at different time points of differentiation. On day 5, a distinct population defined by coexpression of ROR2 and CD13 (II) appeared which was further analyzed for expression of KDR and PDGFRa. b, Quantitative RT-PCR gene expression analysis of the quadruple-positive (III), ROR2+CD13+ (II), and quadruple-negative (I) cells isolated from day-5 embryoid bodies. The average expression is normalized to GAPDH. Mean±S.D., n=3, P<0.05 (one-way analysis of variance (ANOVA)) when comparing populations III to I and II to I. c, Presence of NKX2-5 (left) and MEF2C (right) immunostaining in the QP population 24 hours after sorting and culturing on gelatin-coated plates. Magnification: 200×. d, Immunofluorescence staining of first trimester human hearts revealed pockets of ROR2 positive cells and diffuse KDR and PDGFRα staining in the left ventricle. Magnification: 100×. e, An area of the left ventricle with a cluster of ROR2+ cells that also co-stain with NKX2-5. Magnification: 100×.

FIG. 2. In vitro characterization of quadruple positive cells. a, Immunofluorescence analysis of QP cells 6 days after sorting and cultured on gelatin-coated plates for markers of all three cardiovascular lineages (cardiomyocytes, smooth muscle and endothelial cells). Magnification: 630×. b, Quantitative RT-PCR analysis of OP cells grown in culture after 13 days post-sorting for cardiac genes. c, Upon exposure to 40 ng/ml of VEGF immediately after sorting into Matrigel-coated plates, the QP cells formed a lattice of tubular structures. Magnification: 100×. Endothelial phenotype was further confirmed by Dil-AC-LDL uptake. Magnification: 200×. d, The cells in(c) co-stained for CD31 and von-Willebrand factor. e, Whole-cell voltage clamp recordings of Ca++ transient influx demonstrate ventricular-, atrial-, and pacemaker-like action potentials in the cultured QP population. Magnification: 200×.

FIG. 3. In vivo characterization of quadruple positive cells. a, GFP-hESC-derived OP cells engraft into the peri-infarct regions of mouse hearts. Magnification: 100×. b, Co-staining of GFP with human cardiomyocyte-specific β-myosin heavy chain. Magnification: 100×. c, Myocardial sections from a human fetal heart 6 weeks after heterotopic transplantation into rat abdomen and delivery of QP cells shows clusters of GFP+ cells spread throughout the left ventricle. A similar pattern was observed with transplantation of QP cells into the left ventricles of human fetal hearts engrafted into a mouse ear. Magnification: 200×. d, Co-expression of GFP with cardiac specific markers (α-actinin in the top panel, magnification: 200×, and Troponin in the bottom panel, magnification: 630×) and Connexin43 staining between host and transplanted GFP+ cells. e, GFP+ cells expressing CD31 contiguously with host CD31+ cells. Magnification: 400×. f, Myocardial sections show evoked calcium signals when paced electrically ex vivo. Fluo-4 calcium dye was added to tissue (shown between dashed yellow lines in the gray scale and pseudo colored images) which was then electrically paced at 2 Hz. On the far right panel, the same area after treatment with anti-GFP antibody reveals a GFP₊ area. This region was analyzed for dye intensity changes (f) and results are plotted normalized to the intensity of the initial movie frame (f0). Real time Ca⁺⁺ flux through the tissue indicate functional integration of GFP₊ cells into the host tissue. g, A working model depicting the developmental potential of cardiovascular progenitors (CVP) derived from hESCs based on the four surface markers.

FIG. 4. A schematic representation of the differentiation protocol. a) Embryoid bodies were generated by forced aggregation of H9 cells dissociated into single cells and maintained in TeSR overnight. They were then transferred to StemPro34 media supplemented with Wnt3a (50 ng/ml) for 24 hrs, followed by BMP4, Activin A, and VEGF addition (20 ng/ml each) for 48 hrs. They were subsequently transferred to fresh media containing soluble frizzled-8 (50 ng/ml) and VEGF (10 ng/ml) for an additional 48 hrs. At the end of 5 days, EBs were dissociated into single cells and sorted by expression of ROR2, CD13, KDR, and PDGFR [symbol] . The sorted cells were forced into aggregation again and maintained in a media containing Wnt11 and FGF8 (50 ng/ml each). b) Quantitative RT-PCR analysis of EBs grown according to the protocol outlined above (note that no FACS sorting was performed). Mesoderm and primitive streak-associated genes show a temporal upregulation in the first 5 days of differentiation, while NKX2-5, a cardiac specific gene is enhanced after 5 days. Relative expression is based on comparison between the outlined differentiation protocol and media containing no cytokines. Mean±S.D., n=3.

FIG. 5. Kinetics of ROR2 and CD13 expression based on FACS analysis of differentiating EBs. a) FACS analysis demonstrates the emergence of a ROR2+ population in the first 3 days, followed by co-expression of CD13. On day 5, a distinct ROR2+/CD13+ population develops that also contains KDR/PDGFR [symbol] -expressing cells. b) Quantitative RT-PCR analysis of the three population (I: QN, II: ROR2+/CD13+, and III: QP). While mesoderm and primitive streak associated genes are significantly upregulated in II, cardiac related genes, such as GATA 4 and ISL1 are abundant in the QP cells. The presence of endodermal genes (FOXA2 and SOX17) indicates the impurity of the sorted population. The average expression is normalized to GAPDH. Mean±S.D., n=3, P<0.05 (one-way analysis of variance (ANOVA)) when comparing populations III to I and II to I, except in FOXA2. c) Representative light micrographs of OP cells immediately after sort (right, magnification: 100×) and QP cells grown on gelatin after 7 days (left, magnification: 200×). d) Presence of glandular structures in the QP cells cultured after 3 weeks demonstrating growth of endodermal cells. Magnification: 200×.

FIG. 6. Specification of QP cells into contracting cardiomyocytes. a) Quadruple positive cells were sorted from a GFP-expressing HP cell line and transferred into a synchronous EB generated from wildtype H9 cells. Magnification: 50×. b) The chimeric EBs were observed for an additional 20 days. Beating foci consisting of GFP-positive cells indicated maturation of QP cells into contracting cardiomyocytes. Magnification: 50×. c) When the chimeric Ebs were plated, the GFP-positive cells continued to contract for over 60 days. Magnification: 100×.

FIG. 7. Contribution of cardiomyocytes and endothelial cells. a) A genetically engineered H9 line in which the cardiac troponin T promoter drives GFP expression was used to sort for troponin-positive cells, and a CD31 stain was used to identify endothelial cells. Representative FACS plots from days 10 and 17 post-sort are shown. b) On average, over 57% of sorted cells developed into troponin-expressing cells while endothelial cells contributed to over 20% of cells. Mean±S.D., n=3, *P<0.05 (one-way analysis of variance (ANOVA)). c) The relative number of cardiomyocytes and endothelial cells decreased over time due to overgrowth of the contaminant cells (non-cardiovascular lineage cells).

FIG. 8. Microelectrode array mapping. a) QP sorted cells were grown on a fibronectin coated microelectrode array (MEA). The MEAs each consisted of a 6×6 arrangement of platinum electrodes with 22 μm diameters spaced 100 μm apart. b) Electrical activity was detected from spontaneously beating cultures. Each trace represents data acquired from an individual electrode. c) Application of electrical current through larger electrodes located on the periphery of the MEA was also able to stimulate the QP cultures (right panel) while no electrical activity was noted on QN populations (left panel). d) Combining temporal and spatial information from acquired electrophysiological data enabled 3D maps to be created, demonstrating the propagation of the extracellular action potential. The color-coded activation map demonstrates electrical propagation from the blue region towards the red.

FIG. 9. Transplantation of QP and QN populations into mice. a) Whole mouse heart explanted 8 weeks after injection of GFP+QP cells where the localization of the transplanted cells is visible. b) Transplantation of GFP+QN cells resulted in several localized GFP-positive areas. c) Anti-GFP antibody revealed the presence of QN transplanted cells within the myocardium of the mouse heart. d) Immunohistochemical evidence for teratoma formation after 8 weeks upon transplantation of QN cells. The QN-derived cells gave rise to all three germ layers including columnar epithelium (endoderm, left, magnification: 200×), cartilage (mesoderm, center, magnification: 100×), and neural rosette (ectoderm, right, magnification: 200×).

FIG. 10. Human fetal heart transplantation model. a) Surgical site for implantation of the left ventricle of the human fetal heart in the mouse pinna. The transplanted heart was vascularized and was visibly beating 7-10 days post-engraftment, at which time freshly sorted cells were transplanted. b) Electrocardiographic evaluation of the beating heart in the ear, after background correction for the host native heart, reveals a heart rate of approximately 60 beats per minutes. c) transplantation of an intact late first trimester human fetal heart into rat abdomen. d) Cartoon depicting the surgical anastomosis sites for the human fetal heart transplanted into the murine abdomen. Not shown are the pulmonary veins that are ligated. e) Confocal microscopy evaluation of human fetal heart section 7 weeks after transplantation into the mouse pinna. There is clear connexin 43 staining that connects transplanted hESC-derived quadruple positive cells and the native human cardiomyocytes within the human fetal hearts. On the top, Cx43 is marked with white, Dapi for nuclear staining, and GFP donor cells; on the bottom the same section is shown with overlay of α-actinin staining of both GFP-positive and GFP-negative cells. Magnification: 400×. f) Myocardial sections showing evoked calcium signals when paced electrically ex vivo at 1 Hz. Fluo-4 calcium dye was added to tissue (shown between dashed yellow lines in the gray scale and pseudo colored images) which was then electrically paced. Regions of interest analyzed for dye intensity changes (f) and results are plotted normalized to the intensity of the initial movie frame (f0). On the far left panel, the same area after treatment with anti-GFP antibody reveals a GFP+ area. Real time Ca⁺⁺ flux through the tissue indicate functional integration of GFP+ cells into the host tissue.

### DETAILED DESCRIPTION OF THE EMBODIMENTS

Cardiovascular progenitor cells from in vitro cultures of stem cells are selected for by the method of combining a candidate cell population with reagents that selectively bind to one or more of the markers: ROR2, CD13, KDR and PDGFαR, including all of the markers, and selecting for cells that have bound the reagent. In some embodiments antibodies are used as selective agent(s). Sequential sorting methods may also be employed.

Additional markers for selection include, without limitation, biomolecules present on the cell surface. Such markers include markers for positive selection, which are present on the differentiating cells of interest; and markers for negative selection, which are absent on the differentiating cells of interest, but which typically are present on other cells present in embryoid bodies, e.g. ES cells, endodermal cells, fibroblasts, etc.

Cell compositions obtained by the selective methods of the invention are provided for transplantation of differentiated progenitor cells derived from stem cells, e.g. embryonic stem cells and induced pluripotent cells, usually derived from such stem cells in vitro.

A cell transplant, as used herein, is the transplantation of one or more cells into a recipient body, usually for the purpose of augmenting function of an organ or tissue in the recipient. As used herein, a recipient is an individual to whom tissue or cells from another individual (donor), commonly of the same species, has been transferred. Generally the MHC antigens, which may be Class I or Class II, will be matched, although one or more of the MHC antigens may be different in the donor as compared to the recipient. The graft recipient and donor are generally mammals, preferably human. Laboratory animals, such as rodents, e.g. mice, rats, etc. are of interest for drug screening, elucidation of developmental pathways, etc. For the purposes of the invention, the cells may be allogeneic, autologous, or xenogeneic with respect to the recipient. Cells of interest for transfer include, without limitation, cardiomyocytes and progenitors thereof.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

By “pluripotency” and pluripotent stem cells it is meant that such cells have the ability to differentiate into all types of cells in an adult organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPS cells” it is meant that the differentiated somatic cells can be induced to become, i.e. can be reprogrammed to become, iPS cells. In other words, the somatic cell can be induced to redifferentiate so as to establish cells having the morphological characteristics, growth ability and pluripotency of pluripotent cells. iPS cells have an hESC-like morphology, growing as flat colonies with large nucleo-cytoplasmic ratios, defined borders and prominent nucleoli. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, pluripotent cells are capable of forming teratomas. In addition, they are capable of forming or contributing to ectoderm, mesoderm, or endoderm tissues in a living organism.

Stem Cells and Cultures Thereof.

Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Stem cells of interest also include embryonic cells of various types, exemplified by human iPS and human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shamblott et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection.

Progenitor or Differentiated Cells.

A “differentiated cell” is a cell that has progressed further down the developmental pathway than the cell it is being compared with. Thus, embryonic stem cells can differentiate to lineage-restricted progenitor cells (such as a mesodermal stem cell), which in turn can differentiate into other types of progenitor cells further down the pathway (such as an cardiomyocyte progenitor), and then to an end-stage differentiated cell, which plays a characteristic role in a certain tissue type, and may or may not retain the capacity to proliferate further. For the purposes of the present invention, progenitor cells are those cells that are committed to a lineage of interest, but have not yet differentiated into a mature cell.

The potential of ES cells to give rise to all differentiated cells provides a means of giving rose to any mammalian cell type, and so a range of culture conditions may be used to induce differentiation, including without limitation those conditions set forth herein.

A “cardiomyocyte precursor” is defined as a cell that is capable (without dedifferentiation or reprogramming) of giving rise to progeny that include cardiomyocytes. Such precursors may express various cytoplasmic and nuclear markers typical of the lineage, including, without limitation, cardiac troponin I (cTnI), cardiac troponin T (cTnT), sarcomeric myosin heavy chain (MHC), GATA-4, Nkx2.5, N-cadherin, β1-adrenoceptor (β1-AR), ANF, the MEF-2 family of transcription factors, creatine kinase MB (CK-MB), myoglobin, or atrial natriuretic factor (ANF). Cell surface markers of interest for the selection of cardiomyocyte progenitors include ROR2, CD13, PDGFRα and KDR.

Differentiating Cells.

In the context of cell ontogeny, the adjective “differentiated”, or “differentiating” is a relative term. A “differentiated cell” is a cell that has progressed further down the developmental pathway than the cell it is being compared with. Thus, embryonic stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The potential of ES cells to give rise to all differentiated cells provides a means of giving rose to any mammalian cell type, and so a very wide range of culture conditions may be used to induce differentiation, and a wide range of markers may be used for selection. One of skill in the art will be able to select markers appropriate for the desired cell type.

Among the differentiated cells of interest are cells not readily grown from somatic stem cells, or cells that may be required in large numbers and hence are not readily produced in useful quantities by somatic stem cells.

Cardiomyocyte Lineage Cells.

During normal cardiac morphogenesis, the cranio-lateral part of the visceral mesoderm becomes committed to the cardiogenic lineage. Several heart-associated transcription factors, such as Nkx2.5, Hand1, 2, Srf, Tbx5, Gata4, 5, 6 and Mef2c, become expressed in the cardiogenic region. The first possible overt sign of restriction of gastrulating mesodermal cells to the cardiogenic lineage is the expression of the basic helix-loop-helix transcription factor Mesp1. Cardiogenic mesoderm expressing Mesp1 is pluripotent and contains the precursors for the endocardial/endothelial, the epicardial and the myocardial lineages. The cardiomyocytes of the primary heart tube are characterized by low abundance of sarcomeric and sarcoplasmatic reticular transcripts. Myosin light chain (Mlc) 2v is expressed in a part of the tube that gives rise not only to ventricular chamber myocardium, but also to parts of the atrial chambers and to the atrioventricular node. α and β-myosin heavy chain (Mhc), Mlc1a, 1v and 2a are initially expressed in the entire heart-tube in gradients, and are later restricted to their compartments.

Morphologically and functionally, the chamber myocardium of the developing atria and ventricles are distinguished from the primary myocardium of the linear heart tube. The chamber myocardium becomes trabeculated, whereas the primary myocardium is smooth and covered with cardiac cushions. Markers that in mammals identify the developing chamber myocardium include the atrial natriuretic factor (Anf) and Cx40 genes, which are not expressed in the myocardium of the primary heart tube. During further development, the smooth-walled dorsal atrial wall (comprising the pulmonary and caval myocardium) as well as the atrial septa are incorporated into the atria. These components do not express Anf, but do express Cx40. A gene that is clearly upregulated in the cardiac chambers is sarco-endoplasmic reticulum Ca2+ ATPase (Serca2a), but because it is also expressed in the primary myocardium it is less suited as a marker for the developing chambers. The functional significance of the chamber program of gene expression is that it allows fast, synchronous contractions.

Phenotypes of cardiomyocytes that arise during development of the mammalian heart can be distinguished: primary cardiomyocytes; nodal cardiomyocytes; conducting cardiomyocytes and working cardiomyocytes. All cardiomyocytes have sarcomeres and a sarcoplasmic reticulum (SR), are coupled by gap junctions, and display automaticity. Cells of the primary heart tube are characterized by high automaticity, low conduction velocity, low contractility, and low SR activity. This phenotype largely persists in nodal cells. In contrast, atrial and ventricular working myocardial cells display virtually no automaticity, are well coupled intercellularly, have well developed sarcomeres, and have a high SR activity. Conducting cells from the atrioventricular bundle, bundle branches and peripheral ventricular conduction system have poorly developed sarcomeres, low SR activity, but are well coupled and display high automaticity.

For α-Mhc, β-Mhc and cardiac Troponin I and slow skeletal Troponin I, developmental transitions have been observed in differentiated ES cell cultures. Expression of Mlc2v and Anf is often used to demarcate ventricular-like and atrial-like cells in ES cell cultures, respectively, although in ESDCs, Anf expression does not exclusively identify atrial cardiomyocytes and may be a general marker of the working myocardial cells.

A “cardiomyocyte precursor” is defined as a cell that is capable (without dedifferentiation or reprogramming) of giving rise to progeny that include cardiomyocytes.

Markers.

The markers for selection of cardiomyocyte progenitors according to the present invention include ROR2, PDGFRα, CD13 and KDR.

ROR2, as used herein refers to receptor tyrosine kinase-like orphan receptor 2, which is a predicted 943-amino acid protein with in vitro protein kinase activity, shown in Genbank accession number AAI30523. Many lineage-restricted receptor tyrosine kinases were initially identified as ‘orphans’ homologous to known receptors, and only subsequently used to identify their unknown growth factors. DeChiara et al. (2000) identified one such orphan, encoded by Ror2.

CD13, as used herein refers to aminopeptidase N. The predicted 967-amino acid integral membrane protein has a 24-amino acid hydrophobic segment near its N terminus. Sequence analysis indicated that the hydrophobic segment is not cleaved, but rather serves as both a signal for membrane insertion and as a stable membrane-spanning segment. The remainder of the molecule consists of a large extracellular C-terminal domain that contains a pentapeptide consensus sequence characteristic of members of the zinc-binding metalloproteinase superfamily. Sequence comparisons with enzymes of this class showed that CD13 is identical to aminopeptidase N, an enzyme thought to be involved in metabolism of regulatory peptides by diverse cell types, including small intestinal and renal tubular epithelial cells, macrophages, granulocytes, and synaptic membranes from the central nervous system. The sequence may be accessed at Genbank, NP_{—}001141.

PDGFRα, as used herein may be accessed at Genbank, NP_{—}006197.

KDR, as used herein, refers to the kinaase domain insert receptor. KDR is a receptor for VEGF, and is a type III receptor tyrosine kinase. It functions as the main mediator of VEGF-induced endothelial proliferation, survival, migration, tubular morphogenesis and sprouting. The signalling and trafficking of this receptor are regulated by multiple factors, including Rab GTPase, P2Y purine nucleotide receptor, integrin alphaVbeta3, T-cell protein tyrosine phosphatase, etc. The sequence may be accessed at Genbank, NP_{—}002244.

Specific Binding Member.

The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. Such specific binding members are useful in positive and negative selection methods. Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc.

Especially useful reagents are antibodies specific for markers present on the desired cells (for positive selection) and undesired cells (for negative selection). Whole antibodies may be used, or fragments, e.g. Fab, F(ab′)₂, light or heavy chain fragments, etc. Such selection antibodies may be polyclonal or monoclonal and are generally commercially available or alternatively, readily produced by techniques known to those skilled in the art. Antibodies selected for use will have a low level of non-specific staining and will usually have an affinity of at least about 100 μM for the antigen.

In one embodiment of the invention flow cytometry is used for the selection of cells. In other embodiments methods such as coupling to a magnetic reagent, such as a superparamagnetic microparticle, which antibodies may be referred to as “magnetized” is used.

### Selection of Cells

Differentiating cells of this invention are obtained by culturing or differentiating stem cells in a growth environment that enriches for cells with the desired phenotype. The culture will comprise agents that enhance differentiation to a specific lineage. For example cardiomyocyte differentiation may be promoting by including cardiotropic agents in the culture, such as activin A and/or bone morphogenetic protein-4 (see the Examples herein, Xu et al. Regen Med. 2011 January; 6(1):53-66; Mignone et al. Circ J. 2010 74(12):2517-26; Takei et al. Am J Physiol Heart Circ Physiol. 2009 296(6):H1793-803, each herein specifically incorporated by reference). Examples of such protocols also include, for example, addition of a Wnt agonist, such as Wnt 3A, optionally in the presence of cytokines such as BMP4, VEGF and Activin A; followed by culture in the presence of a Wnt antagonist, such a soluble frizzled protein (as described in the Examples). However, any suitable method of inducing cardiomyocyte differentiation may be used, for example, Cyclosporin A described by Fujiwara et al. PLoS One. 2011 6(2):e16734; Dambrot et al. Biochem J. 2011 434(1):25-35; equiaxial cyclic stretch, angiotensin II, and phenylephrine (PE) described by Foldes et al. J Mol Cell Cardiol. 2011 50(2):367-76; ascorbic acid, dimethylsulfoxide and 5-aza-2′-deoxycytidine described by Wang et al. Sci China Life Sci. 2010 53(5):581-9, endothelial cells described by Chen et al. J Cell Biochem. 2010 111(1):29-39, and the like, which are herein specifically incorporated by reference.

The cells are harvested at an appropriate stage of development, which may be determined based on the expression of markers and phenotypic characteristics of the desired cell type e.g. at from about 1 to 4 weeks. Cultures may be empirically tested by staining for the presence of the markers of interest, by morphological determination, etc. The cells are optionally enriched before or after the positive selection step by drug selection, panning, density gradient centrifugation, etc. In another embodiment, a negative selection is performed, where the selection is based on expression of one or more of markers found on ES cells, fibroblasts, epithelial cells, and the like. Selection may utilize panning methods, magnetic particle selection, particle sorter selection, and the like.

For positive or negative selection, separation of the subject cell population utilizes affinity separation to provide a substantially pure population. Techniques for affinity separation may include flow cytometry, magnetic separation using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Any technique may be employed which is not unduly detrimental to the viability of the selected cells.

Specific binding members, usually antibodies, are added to the suspension of cells, and incubated for a period of time sufficient to bind the available antigens. The incubation will usually be at least about 2 minutes and usually less than about 30 minutes. It is desirable to have a sufficient concentration of antibodies in the reaction mixture so that the efficiency of the magnetic separation is not limited by lack of antibody. The appropriate concentration is determined by titration.

The suspension of cells is applied to a separation device, and sorted for expression of the markers of interest. The cells may be collected in any appropriate medium. Various media are commercially available and may be used according to the nature of the cells, including dMEM, HBSS, dPBS, RPMI, PBS-EDTA, PBS. Iscove's medium, etc., frequently supplemented with fetal calf serum, BSA, HSA, etc.

The composition of selected cells is enriched for the desired cell type or lineage. Usually at least about 50% of the total cells in the population will be the selected differentiating cells, more usually at least about 75% of the cells, and preferably at least about 90% of the cells, at least about 95% of the cells, or more.

The compositions thus obtained have a variety of uses in clinical therapy, research, development, and commercial purposes. For therapeutic purposes, for example, cardiomyocytes and their precursors may be administered to enhance tissue maintenance or repair of cardiac muscle for any perceived need, such as an inborn error in metabolic function, the effect of a disease condition, or the result of significant trauma.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or β-galactosidase); that have been prelabeled (for example, with BrdU or [³H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Where the differentiating cells are cells of the cardiomyocyte lineage, suitability can also be determined in an animal model by assessing the degree of cardiac recuperation that ensues from treatment with the differentiating cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N₂ on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

The differentiated cells may be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. The cells may be administered to a recipient heart by intracoronary injection, e.g. into the coronary circulation. The cells may also be administered by intramuscular injection into the wall of the heart.

Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

The differentiating cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5, and MEF2-C.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20):11939-44).

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells.

The vectors may include genes that must later be removed, e.g. using a recombinase system such as Cre/Lox, or the cells that express them destroyed, e.g. by including genes that allow selective toxicity such as herpesvirus TK, bcl-xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

The cells of this invention can be used to prepare a cDNA library relatively uncontaminated with cDNA preferentially expressed in cells from other lineages. For example, cardiomyocytes are collected by centrifugation at 1000 rpm for 5 min, and then mRNA is prepared from the pellet by standard techniques (Sambrook et al., supra). After reverse transcribing into cDNA, the preparation can be subtracted with cDNA from undifferentiated ES cells, other progenitor cells, or end-stage cells from the cardiomyocyte or any other developmental pathway.

The differentiated cells of this invention can also be used to prepare antibodies that are specific for markers of cardiomyocytes and their precursors. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used to identify or rescue cells of a desired phenotype from a mixed cell population, for purposes such as costaining during immunodiagnosis using tissue samples, and isolating precursor cells from terminally differentiated cardiomyocytes and cells of other lineages.

Of particular interest is the examination of gene expression in the differentiating of the invention. The expressed set of genes may be compared against other subsets of cells, against ES cells, against adult heart tissue, and the like, as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of polypeptide of interest. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

The cells are also useful for in vitro assays and screening to detect factors that are active on differentiating cells, including cells of the cardiomyocyte lineage. Of particular interest are screening assays for agents that are active on human cells. A wide variety of assays may be used for this purpose, including immunoassays for protein binding; determination of cell growth, differentiation and functional activity; production of factors; and the like.

In screening assays for biologically active agents, viruses, etc. the subject cells, usually a culture comprising the subject cells, is contacted with the agent of interest, and the effect of the agent assessed by monitoring output parameters, such as expression of markers, cell viability, and the like. The cells may be freshly isolated, cultured, genetically altered as described above, or the like. The cells may be environmentally induced variants of clonal cultures: e.g. split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:I to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers.

For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

The composition may optionally be packaged in a suitable container with written instructions for a desired purpose, such as the reconstitution of cardiomyocyte cell function to improve some abnormality of the cardiac muscle.

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

### Example 1

A staged protocol was developed where H9 human embryonic stem cells (hESC) were differentiated towards cardiac mesoderm. Embryoid bodies (EB) were formed by forced aggregation of single hESCs and were exposed to Wnt3a at 10 ng/ml for the first 24 hrs in a serum free media (StemPro). They were then exposed to BMP4 (10-20 ng/ml), Activin (5 ng/ml) and bFGF (5 ng/ml) for the next 3 days. On day 4.5, the EBs were dissociated and stained with our candidate markers: ROR2 (with PE secondary), CD13 (conjugated to APCCy7), KDR (conjugated to 607), and PDGFRa (conjugated to biotin with a 605 streptavidin secondary). The stained cells were sorted for the quadruple positive populations (ROR2+/CD13+/KDR+/PDGFRa+). The sorted cells were then plated in an ultra-low attachment V shape 96 well and spun down at 500 rpm for 30 seconds to aggregate the cells. 24 hours later, the cells had grown and were transferred to a gelatin coated tissue culture plate. These cells were also subjected to field stimulation and spontaneous beating were observed after 8-12 days. We showed that the negative population failed to give rise to any beating phenotype.

The freshly sorted GFP+ quadruple+cells were transplanted into the mouse model heart. Left coronary artery was ligated, an ischemic area was created and the cels were transplanted into the peri-infarct area of a NOG mouse heart. After 8 weeks, the heart tissue was harvested and it was shown that the candidate progenitors were engrafted and matured to cardiomyocytes.

Furthermore, we stained 1st and 2nd trimester human fetal heart tissue with the antibodies for our candidate markers. We showed that there are areas ROR2+ and PDGFRa+ within the fetal heart. No CD13+ areas were observed.

### Example 2

### Prospective Isolation of Human Embryonic Stem Cell-Derived Cardiovascular Progenitors that Integrate into the Human Fetal Heart

A goal of regenerative medicine is to identify cardiovascular progenitors from human embryonic stem cells (hESC) that can functionally integrate into the human heart. Prior studies to evaluate the developmental potential of candidate hESC-derived progenitors have delivered these cells into murine and porcine cardiac tissue, with inconclusive evidence regarding the capacity of these human cells to physiologically engraft in xenotransplantation assays. Further, the potential of hESC-derived cardiovascular lineage cells to functionally couple to human myocardium remains untested and unknown. Here, we have prospectively identified a population of hESC-derived ROR2+/CD13+/KDR+/PDGFR [symbol] + (quadruple positive, or QP) cells that give rise to cardiomyocytes, endothelial cells, and vascular smooth muscle cells in vitro. We observed rare clusters of ROR2+ cells and diffuse expression of KDR and PDGFR [symbol] in first trimester human fetal hearts. We developed a novel in vivo transplantation model by heterotopically transplanting first trimester human fetal hearts into the abdomen of nude rats via large vessel anastomosis, which contracted rhythmically for up to 8 weeks. The QP cells were then delivered into the left ventricle of these intact, beating fetal hearts. In contrast to traditional murine heart models for cell transplantation, we show structural and functional integration of hESC-derived cardiovascular progenitors into human hearts.

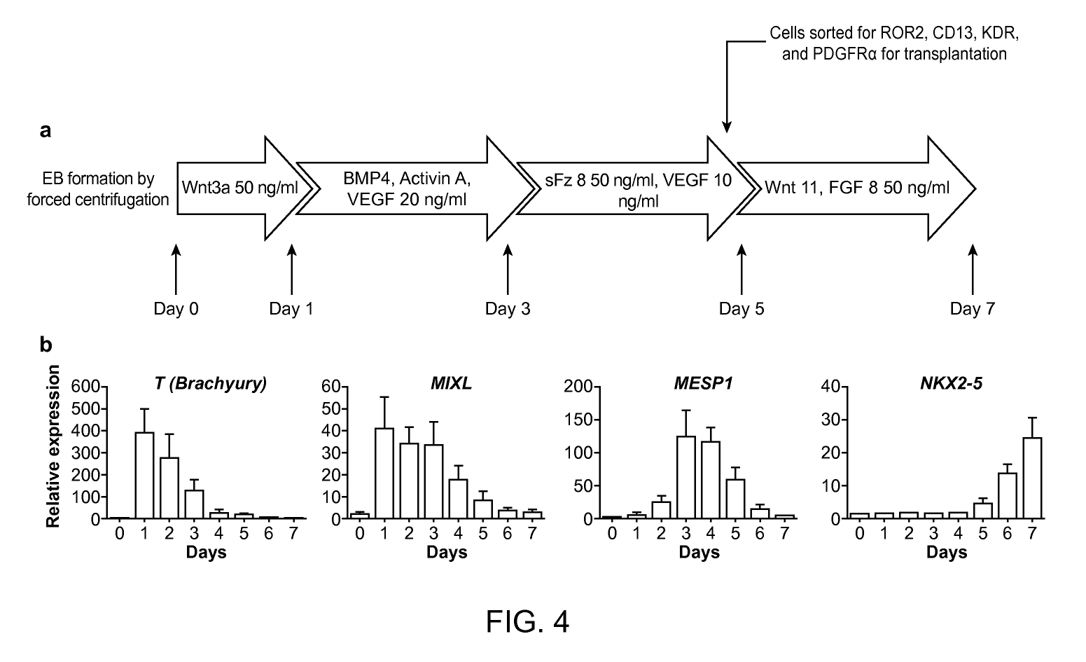

The need for enrichment of cardiovascular lineage cells from hESCs has led to myriad research strategies utilizing chemical, genetic, epigenetic, and lineage selection strategies to direct cardiac differentiation. Despite these efforts, directed differentiation results in a heterogeneous population whose contaminants include undifferentiated cells capable of forming teratomas. To differentiate hESCs efficiently into cardiovascular lineages, we established a protocol based on stage-specific activation and then inhibition of canonical Wnt/β-catenin pathway in the hBCL2-hESC line, with temporal addition of activin A, BMP4, VEGF, and FGF8 (FIG. 4) (enforced expression of the Bcl2 gene in this transgenic line greatly improves the survival of hESCs upon manipulation). Despite this robust differentiation assay, many cells with noncardiovascular developmental fates remained in the final product.

To enrich or isolate a population of cardiovascular progenitors from hESCs based on surface markers we screened a large panel of monoclonal antibodies (mAbs). A member of the receptor tyrosine kinase-like orphan receptor family, ROR2, and an aminopeptidase-N, CD13, individually were shown to enrich cardiovascular progenitors, along with Flk-1 (KDR) and platelet-derived growth factor-α (PDGFRα).

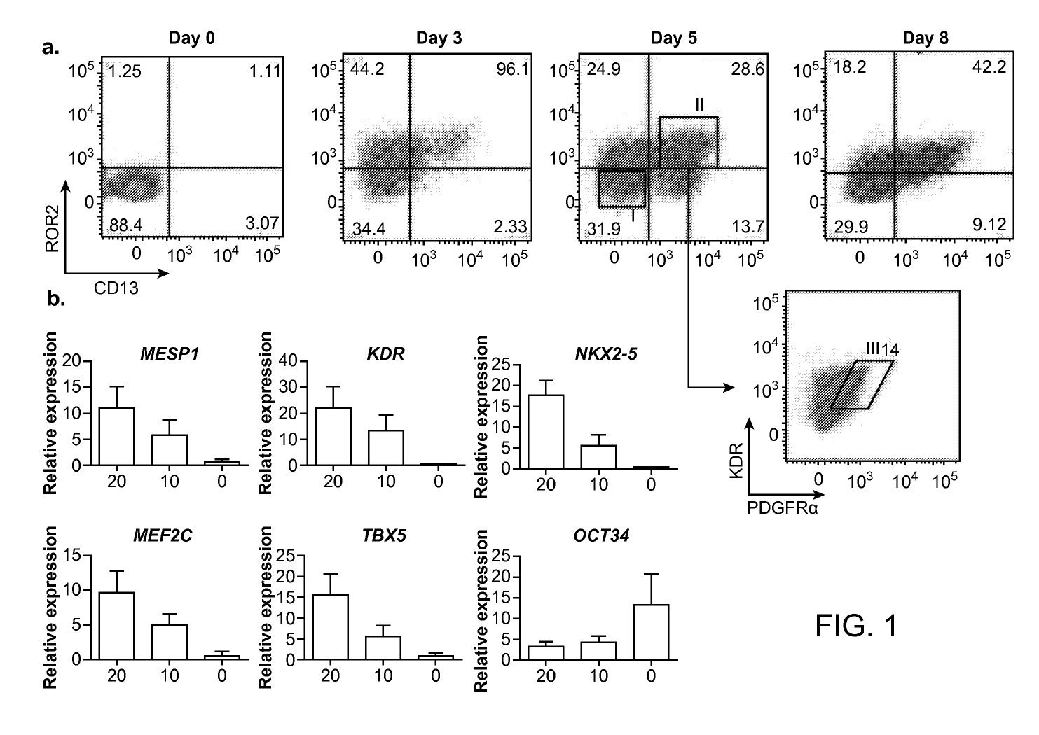

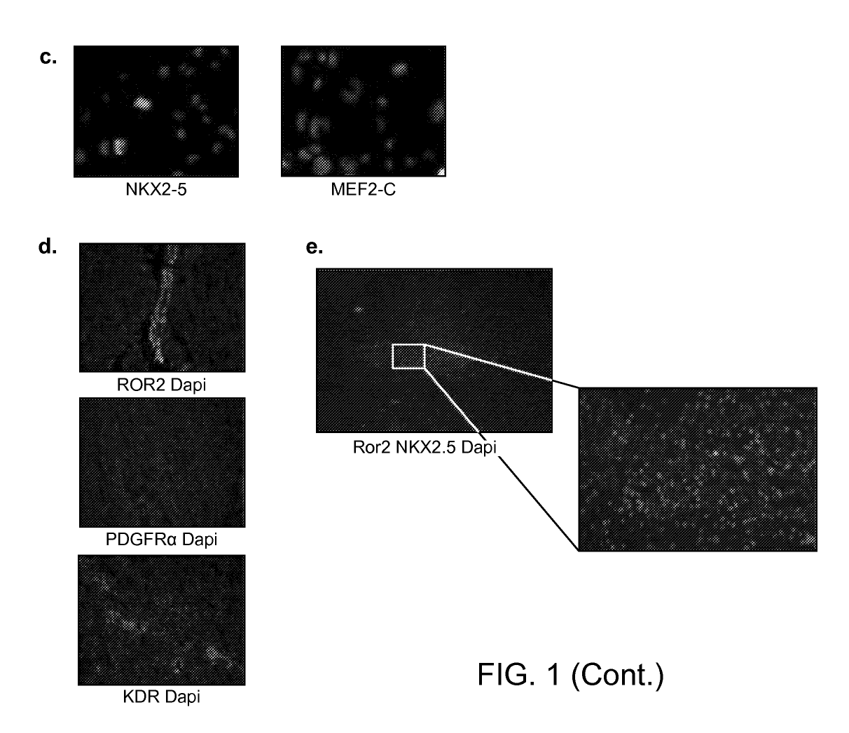

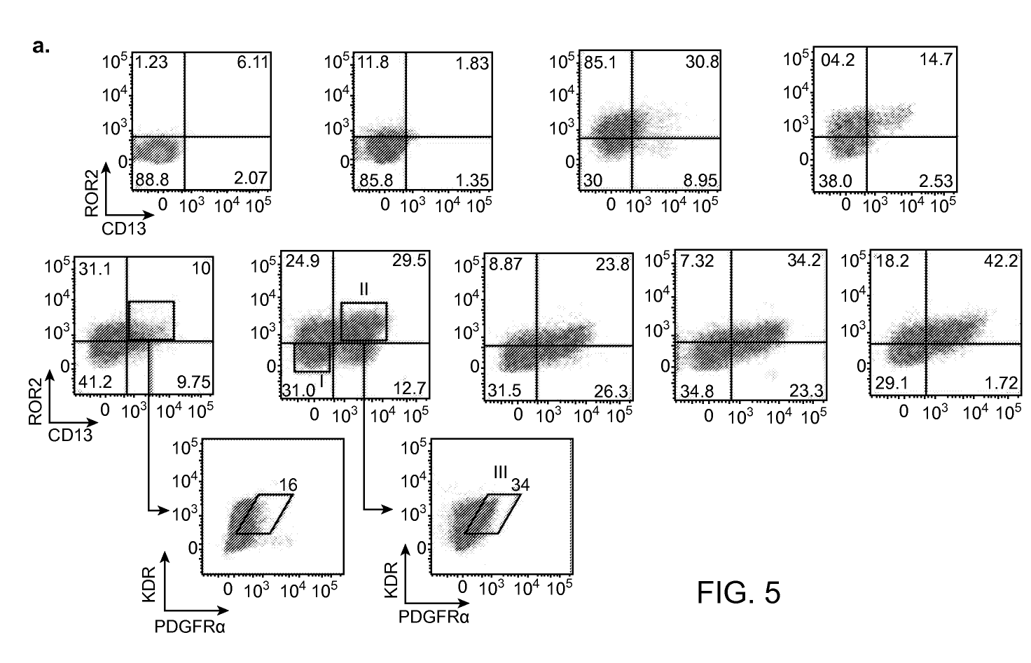

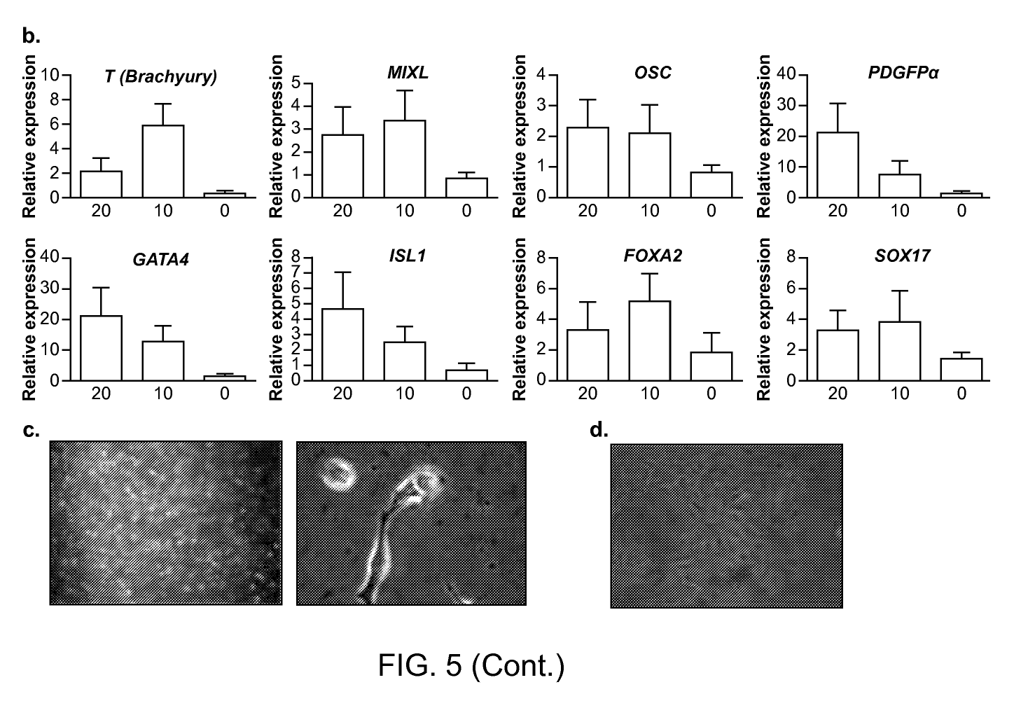

To determine whether cardiovascular progenitors develop from a subpopulation of differentiating hESCs that expresses one or more of these surface markers, we analyzed embryoid bodies (EBs) after 5 days of differentiation for expression of ROR2, CD13, KDR, and PDGFR-α. As shown in FIG. 1a and FIG. 5a, a distinct population marked by co-expression of ROR2 and CD13 emerges temporally as hESCs differentiate. This population exhibited a transcriptional profile similar to primitive streak/mesodermal cells (FIG. 5b). The ROR2+/CD13+ population was sorted and expression of KDR and PDGFRα was examined and confirmed.

We then evaluated the lineage commitment of the ROR2+/CD13+/KDR+/PDGFRα+ population (hereafter referred to as quadruple positive, or QP, population). The QP population expressed high levels of cardiac mesoderm and cardiac development genes, including MESP1 (mesoderm posterior 1), the earliest known marker for cardiogenesis, and key cardiac transcription factors of the primary and secondary heart fields, including TBX5, GATA4, MEF2C, NKX2.5, and ISL1 (FIG. 1b and FIG. 5b). In contrast, the fraction of cells in the EBs that was negative for all four markers had the highest expression of pluripotency genes, indicative of residual undifferentiated cells. Although the QP population exhibited high expression of cardiac lineage genes, it also expressed genes corresponding to primitive-steak and endodermal cells, though to a much lesser degree (FIG. 5b). Enrichment for cardiac lineage cells in the majority of the sorted QP population was confirmed by protein-level detection of TBX5, MEF2C, and GATA4 (FIG. 1c). The expression pattern of these four surface markers during early human fetal heart formation was determined by immunohistochemistry to elucidate their expression during in utero development. KDR and PDGFRa were broadly expressed in 9 to 10 week-old human fetal cardiac tissue, including the vasculature.

Rare distinct areas of ROR2 expression were detected in the myocardium and interventricular septum, but not in the epicardium (FIG. 1d). In contrast, we did not detect any evidence of CD13 expression. The role of the ROR2 protein has been studied in developmental processes, cell migration, and polarity. It has been shown that ROR2 is expressed in the entire primitive streak region during mouse embryonic development and later in the developing limbs, brain, heart and lungs25. In fact ROR2-deficient mice die within 6 hours of birth, demonstrating dwarfism, short limbs, and cyanosis. The observed cyanosis and early postnatal death is partially attributed to a ventricular septal defect detected in the mutant mice. In humans, mutations in the ROR2 gene have been associated with autosomal recessive Robinow syndrome, characterized by short stature, mesomelic limb shortening, abnormal craniofacial features, and distinct cardiac anomalies affecting the myocardium.

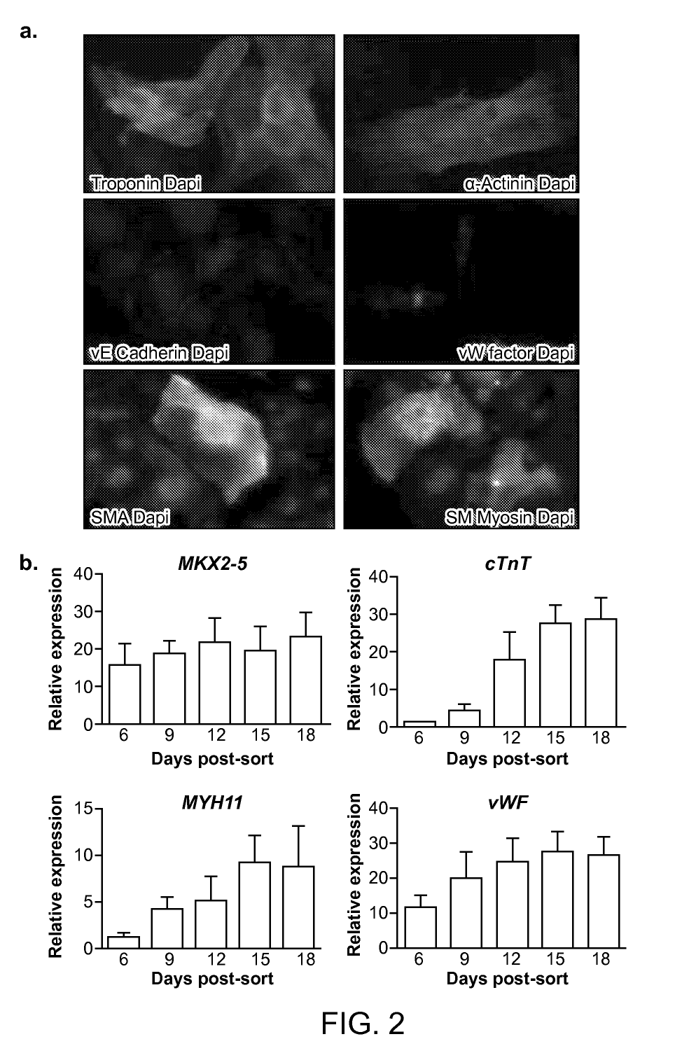

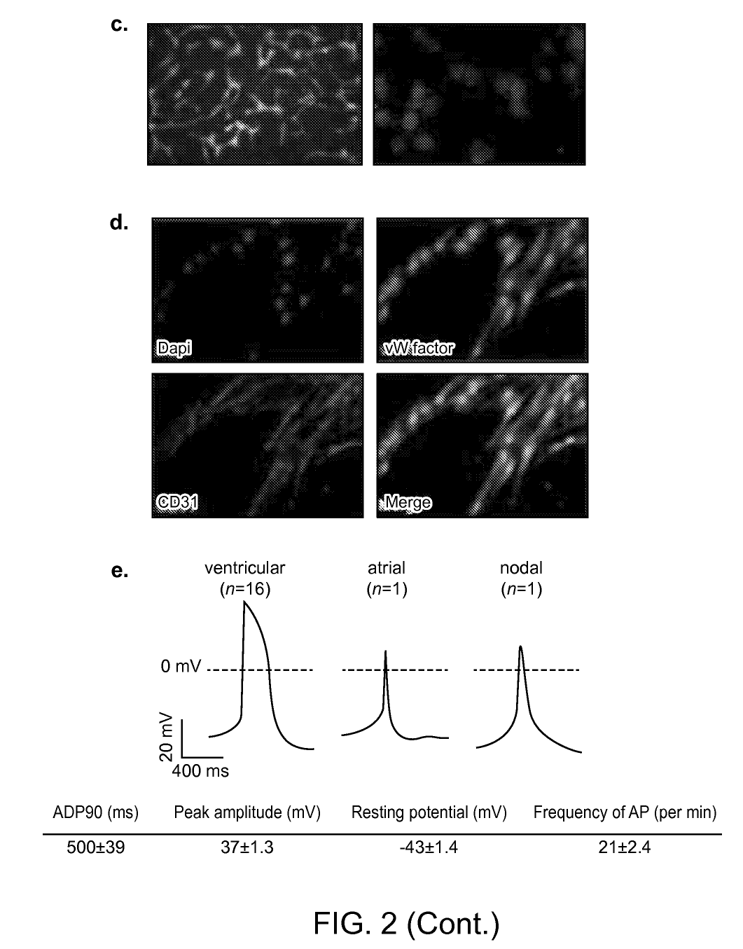

After confirmation of the embryonic developmental relevance of the majority of the markers, we set out to further characterize the in vitro developmental potential of the OP progenitor population. Freshly-sorted QP cells were cultured as aggregates in suspension for an additional 7-10 days in the presence of Wnt11 and FGF8 in serum-free media. Consistent with the gene expression profile described above, the QP population gave rise to cells of the cardiovascular lineage based on immunostaining and gene expression (FIG. 2a). This progenitor population is multipotent and able to generate cardiomyocytes as well as smooth muscle cells and endothelial cells, the hallmark downstream lineages of cardiovascular progenitors. We consistently detected a high frequency of cardiomyocytes beating spontaneously as a synchronous mass.

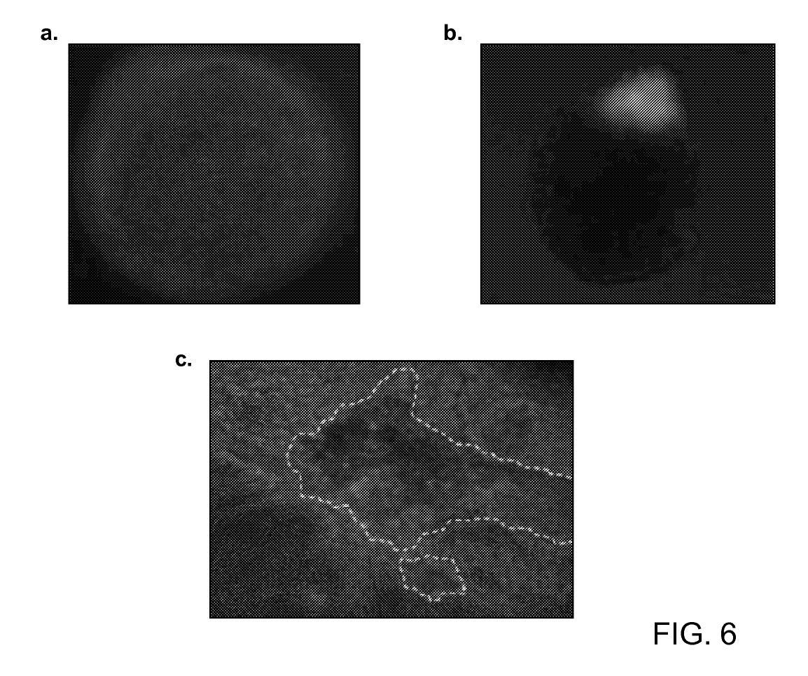

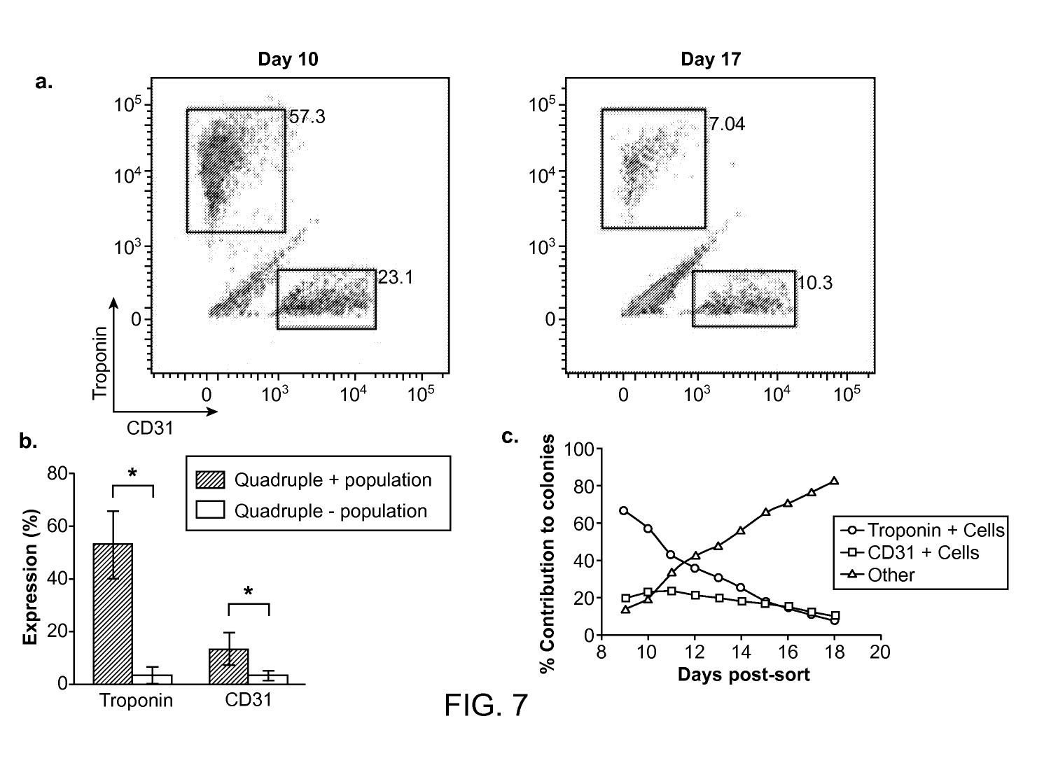

We next confirmed the specification of the QP population to a cardiomyocyte fate by transferring freshly sorted ROR2+/CD13+/KDR+/PDGFRα+ cells derived from a GFP-expressing hESC line into a synchronous embryoid body derived from unlabelled hESCs. The majority of the GFP+ cells in the chimeric EB developed into contracting foci (FIG. 6). To quantify the extent of cardiomyocyte generation, we used a cardiac troponin-GFP reporter hESC line and maintained the QP population in culture for over 30 days. Over 77% of the derived cells were cardiomyocytes or endothelial cells, based respectively on troponin and CD31 expression, reflecting efficient enrichment of progenitors in the QP population (FIG. 7).

Immunohistochemistry of the QP population 10 days after sorting demonstrated cells that expressed: CD31, von Willebrand factor, and VE-Cadherin, indicative of endothelial cells; troponin, α-actinin, and myosin heavy chain, indicative of cardiomyocytes; and smooth muscle actin and smooth muscle myosin, indicative of smooth muscle cells. When plated on Matrigel coated dishes and treated with a high concentration of VEGF, the QP cells acquired the morphology of endothelial cells and formed a lattice. These cells expressed CD31 and von Willebrand factor and efficiently incorporated Dil-AC-LDL, confirming their endothelial phenotype functionally (FIG. 2a-e).

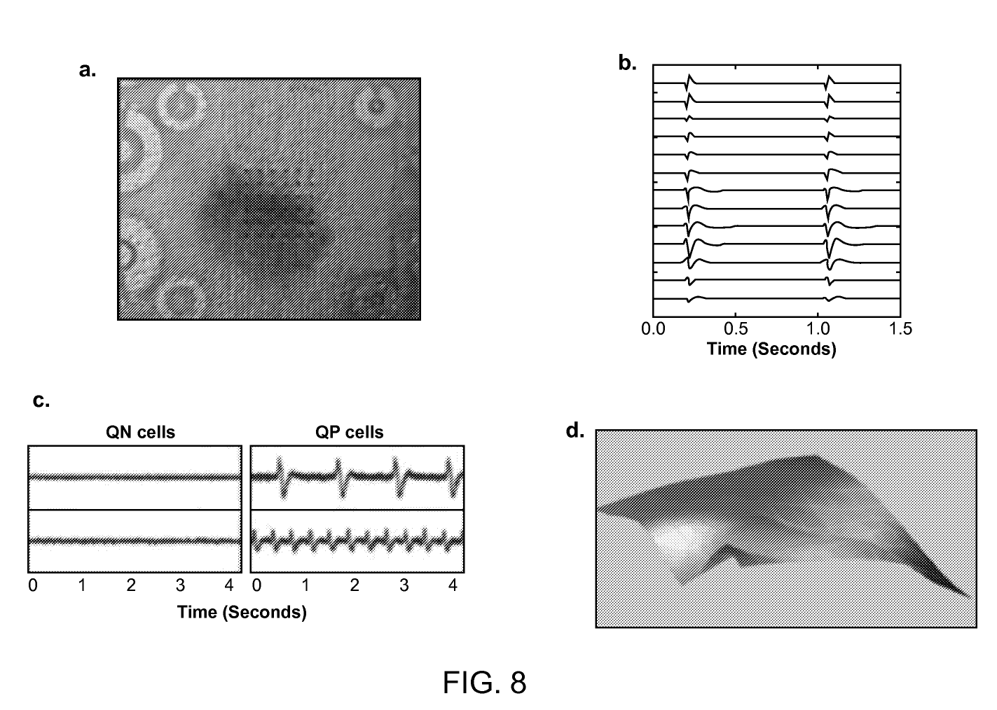

The functional properties of the QP-derived cardiomyocytes were evaluated 10 days after sorting, using field potential measurements, as well as whole-cell patch clamp (FIG. 2f). Synchronous multifocal field potential recordings performed on microelectrode arrays showed electrical activity throughout the adherent cultures (FIG. 8). Mapping of the delay of time points of maximal downstroke velocity at each electrode revealed a homogenous spread of excitation. Additionally, action potential (AP) recordings from single cells revealed the presence of pacemaker-, atrial-, and ventricular-like patterns characterized predominantly by a fast phase 1 depolarization. More than 90% of the single cells studied exhibited a ventricular-like AP morphology. These results confirm that the QP population can differentiate to contractile cardiomyocytes with a fetal-like AP phenotype.

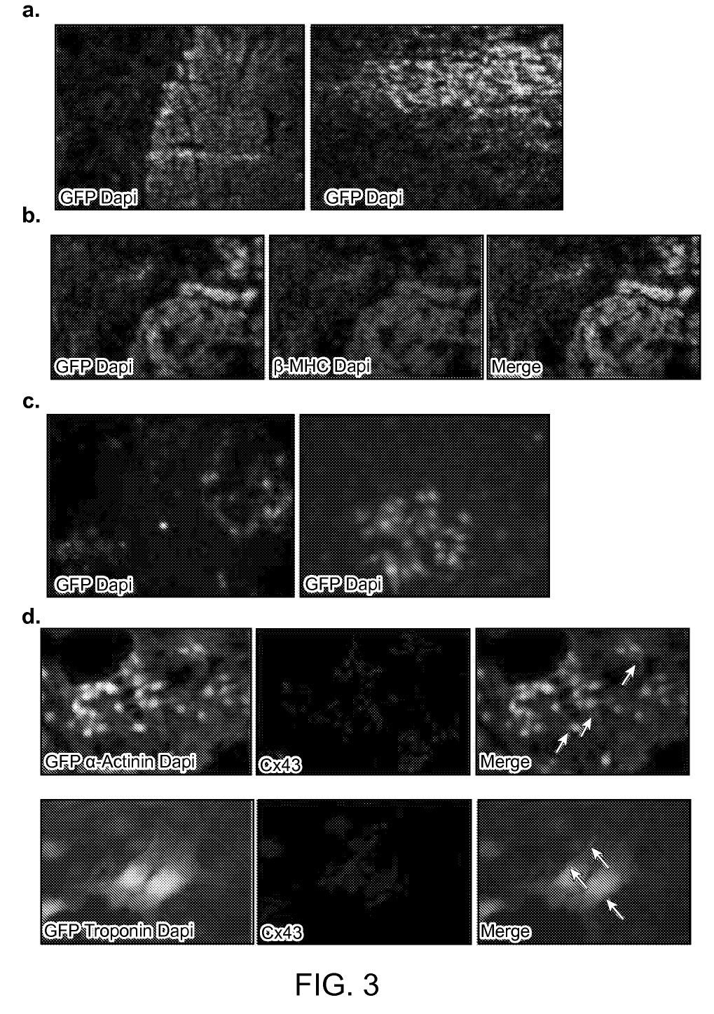

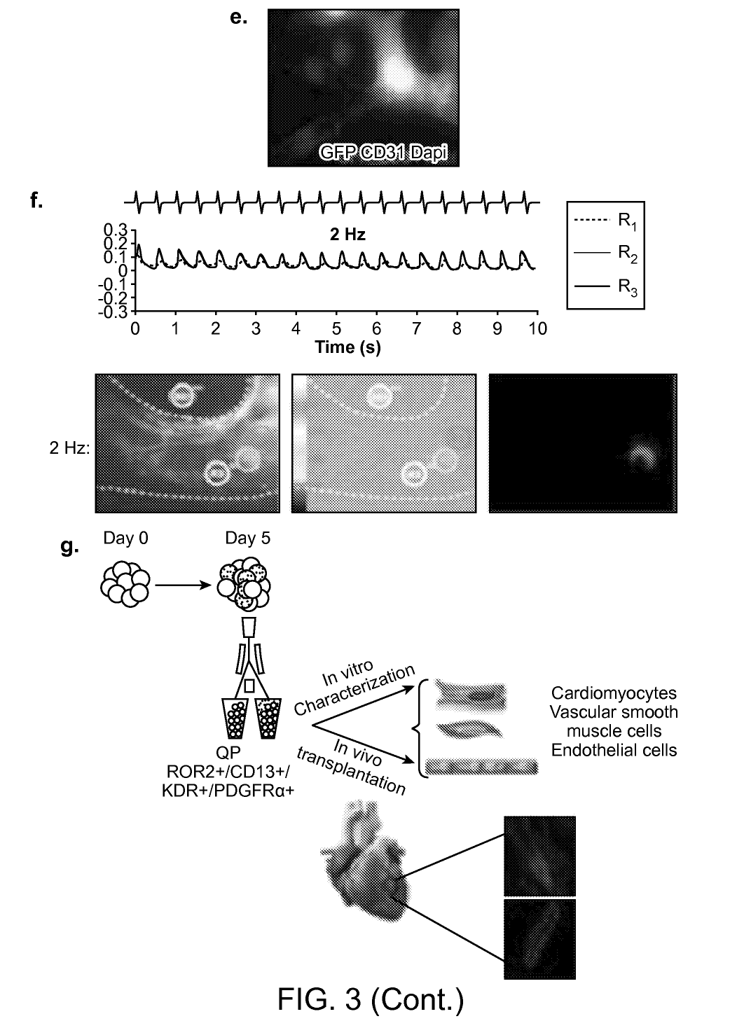

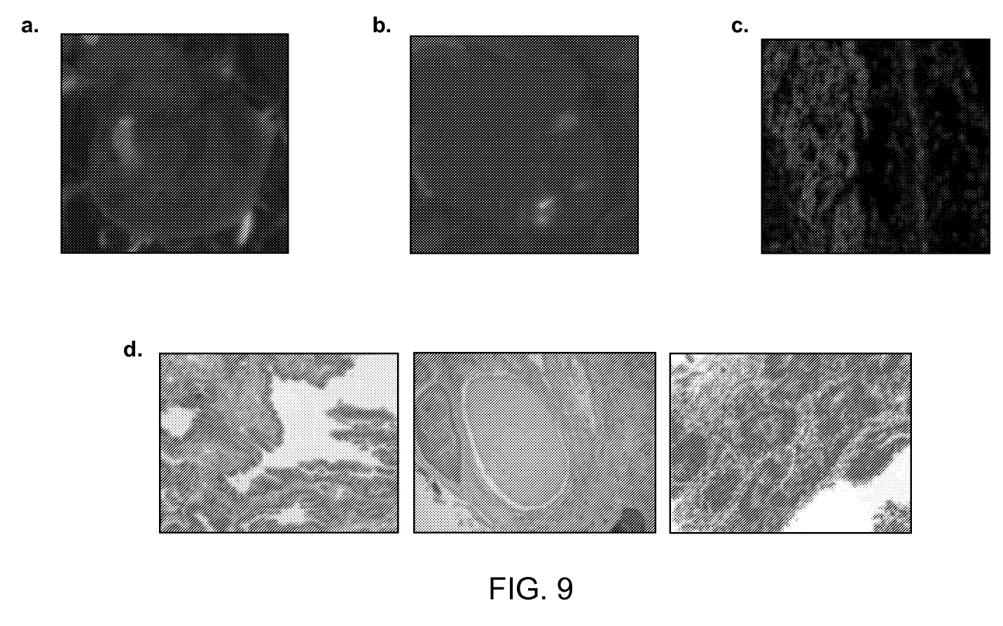

To test their in vivo developmental potential, day-5 EB-derived QP cells from a GFP-hESC line were transplanted into the healthy or injured hearts of non-obese diabetic/severe combined immunodeficient mice with common gamma chain knockout (NSG). Approximately 5-10×10⁵ ROR2/CD13/KDR/PDGFRα-positive cells were sorted and immediately transplanted by direct injection into the left ventricle of healthy mice or into the pen-infarct area of mice following occlusion of the left anterior descending artery (LAD). As controls, quadruple-negative (QN) cells from the same EB culture as above were also sorted and transplanted in similar areas in healthy and injured NSG mice. The animals were euthanized after 8 weeks and histological analyses of the explanted OP-transplantation hearts showed clusters of GFP-positive cells throughout the injected area (FIG. 3a, b). While no teratomas were observed in any of the animals transplanted with the OP cardiovascular progenitors, one out of the seven mice transplanted with the QN cells developed teratomas in the heart (FIG. 9), demonstrating that even day-5 EB cells harbor some teratogenic cells. The GFP-positive OP cells were detected only as clusters in the injection sites with no significant migration, but exhibited cardiac differentiation as evidenced by expression of myosin heavy chain (FIG. 3b).

Despite their engraftment and differentiation, detailed histological examination of the explanted hearts showed no gap junction formation between hESC-derived cardiovascular progenitors and the host myocardium: the QP cells failed to integrate with the mouse myocardium. We hypothesized that the failure of hESC derived cardiomyocytes to structurally and functionally integrate into the adult mouse host may be due to several factors including: i) interspecies differences that prevent the coupling of human and mouse cells, ii) inability of an adult heart to provide an optimal environment for maturation and integration of the cardiac progenitors, and/or iii) an inherent inability of QP-derived cardiovascular progenitors to functionally integrate. These issues have been difficult to address in the absence of an in vivo human heart model that would allow long-term assessment of the integration of hESC-derived cells.

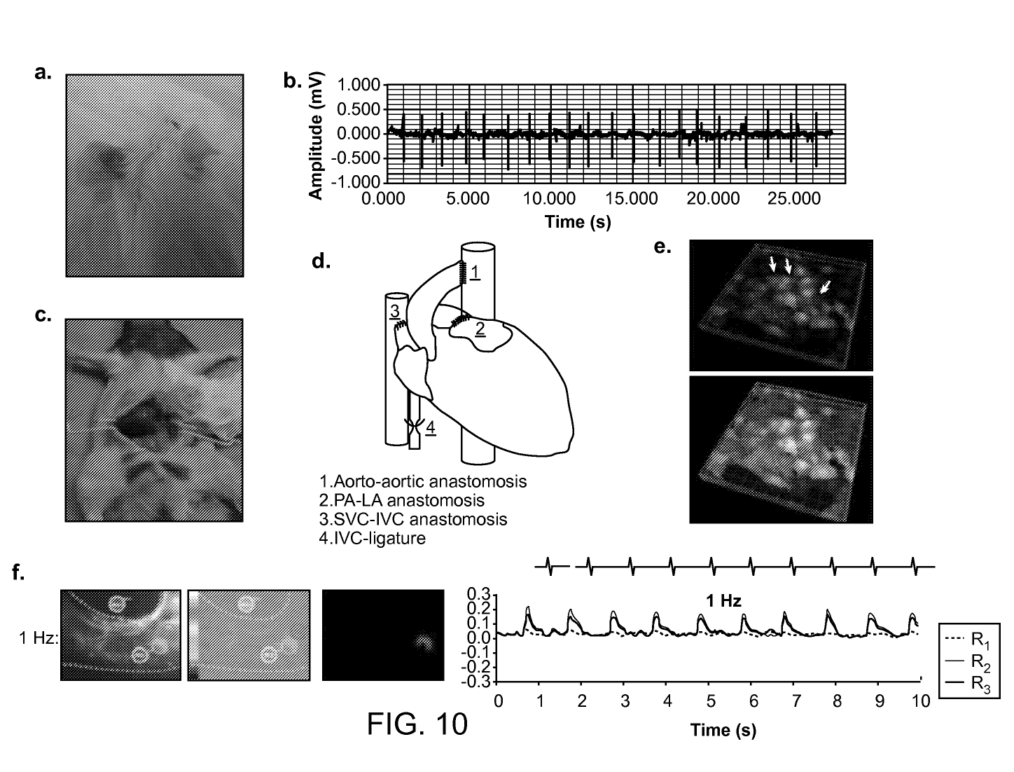

Thus, we developed two novel transplantation models, which allow us to assess the functional development of hESC derived cardiovascular progenitors in fetal human hearts. In the first model, the ventricles from a 1st trimester human fetal heart (7 weeks) were implanted subcutaneously into a pouch formed in the ear pinna of a SCID mouse (FIG. 10a, b). Graft viability was confirmed by the presence of autonomous beating determined by visual inspection and electrocardiography approximately 7-10 days after implantation. Two weeks later, approximately 5×10⁵ freshly sorted QP cardiovascular progenitors (and ON cells as control) from a GFP-hESC line were transplanted into the heart graft. Developmental potential of the hESC-derived OP cells was assessed 8 weeks after transplantation by immunohistochemistry.

Although the ear-heart graft represents a viable cardiac tissue, it provides no physiological activities (i.e. hemodynamics or sinoatrial and atrioventricular conduction), the lack of which may influence the development of hESC-derived cardiovascular progenitors. To circumvent these shortcomings, in the second model a late 1st trimester human fetal heart (11 weeks) was transplanted into the abdomen of an immunodeficient mouse or rat. The intact aorta from the human fetal heart was anastomosed to the rodent's abdominal aorta, the superior vena cava was sutured onto the rodent's inferior vena cava, and the human pulmonary artery and left atrium were anastomosed (FIG. 10c, d). Upon release of the cross-clamp, the human fetal heart began regular and rhythmic contractions, with coronary perfusion and blood flow throughout all chambers. This viable and functioning human heart exhibits physiologic hemodynamics, as well as sinoatrial and atrioventricular conduction. Therefore, it offers a physiologically-competent system to assess the fates of transplanted hESCderived cardiovascular progenitors in human hearts.

Seven to ten days after heart transplantation, the abdominal wall was opened and approximately 5×10⁵GFP-hESC-derived QP or QN cells were directly injected into the left ventricle of the human fetal heart. The animals were euthanized after 8 weeks, and confocal microscopy of the explanted hearts receiving QP cells revealed clusters of GFP-positive cells spread throughout the myocardium, including areas distant from the injection site. It is not clear whether the distribution of grafted cells is due to migration or simple spreading of the cells along the injection site; however, the absence of engrafted cells distal from the injection site in the mouse heart transplantation argues against spreading. The GFP-positive cells co-expressed troponin, a-actinin, and CD31, which implies in vivo differentiation of the progenitors into cardiomycytes and endothelial lineages (FIG. 3c-e, FIG. 10e). In contrast, transplanted QN cells did not differentiate into cardiomyocyte or endothelial lineages. A careful histological examination of the explanted QP-recipient hearts revealed typical punctate staining for Connexin-43 along the regions of intimate cell-to-cell contact between hESC derived cardiomyocytes and host cardiomycytes, which was not observed in murine xenograft models (FIG. 3c). These results indicate that when transplanted into human fetal hearts, hESC derived cardiovascular progenitors not only mature to cardiomycytes, they also couple structurally to their neighboring cells.

To determine whether the transplanted cells were electrically connected to the host myocardium, heterotopic human fetal hearts were removed from the rat abdomen, immediately sectioned, and real-time Ca⁺⁺ transients were measured in areas with QP-derived GFP-positive cells. GFP-positive cells demonstrated periodic Ca⁺⁺ oscillations similar to and synchronized with the host cells. The Ca⁺⁺ oscillations responded to increasing frequencies of external electrical stimulation. These recordings showed conduction of Ca⁺⁺ signals from the host myocardium into areas of GFP-positive transplanted cells resembling a continuous electrical propagation (FIG. 3f, FIG. 10f). Taken together, these data demonstrate that hESC-derived cardiovascular progenitors, defined by four surface markers, can structurally and functionally integrate into the electrical syncytia of a human fetal heart upon transplantation.

This is the first report of engraftment, maturation, and integration of hESC-derived cardiovascular progenitors into human hearts. Additionally, our finding of ROR2 as an early marker for cardiac lineage specification highlights a previously unknown role of ROR2 expression in cardiac development. These valuable results provide the basis for hESC based cardiac therapy by identification of a progenitor population capable of engraftment and regeneration without risk of teratoma formation.

### Methods

Human ES cell culture and differentiation. Human ES cell lines H9 and H7 (WiCell Research Institute, NIH code WA09 and WA07, respectively) were maintained according to standard protocols. The cardiac troponin-GFP reporter H9 ES line was a generous gift from Dr. Timothy Kamp's laboratory. Constitutive enhanced GFP-expressing H9 ES cells were generated as described previously using Lentilox3.7 (Science gateway). Human ESCs were later maintained on feeder-free conditions using growth-factor depleted Matrigel- (BD Biosciences) coated plates with mTeSR media (Stemcell Technologies). When confluent, hESCs were dissociated into single cells using Accutase (Sigma) for generation of embryoid bodies. Approximately 2,000 hESCs in 100 μl of mTeSR media was seeded into each well of a 96-well V-shape bottom ultra low attachment plate (Corning) and centrifuged at 300×g for 3 minutes. After incubation overnight, the EBs were transferred to six-well ultra-low attachment plates in 2.5 ml of StemPro34 (Invitrogen) containing 2 mM glutamine (Gibco), 4×10⁻⁴ M monothioglycerol (MTG) (Sigma), 150 μg/ml transferrin (Roche), and 50 μg/ml ascorbic acid (Sigma). To induce differentiation towards the cardiovascular lineage, the following cytokines were added: day 0-1, Wnt3a (50 ng/ml); days 1-3, BMP4 (20 ng/ml), VEGF (20 ng/ml), Activin A (20 ng/ml); days 3-5, soluble Frizzled-8 (sFz8, 50 ng/ml), VEGF (10 ng/ml); days 5-7, FGF8 (50 ng/ml) and Wnt 11 (50 ng/ml). Cultures were incubated in a 5% CO₂/5%/O₂/90% N2 environment. Wnt3a and sFz8 were generous gifts from Dr. Roel Nusse's lab, BMP4, VEGF, Activin A, and Wnt11 were obtained from R&D Systems, FGF8 was purchased from Pepprotech.

Flow Cytometry and sorting. Embryoid bodies were dissociated using Accutase and passed through a cell strainer (40 μm pore size) to obtain single cells. Dissociated cells were stained with antibodies against ROR2 (unconjugated), CD 13, PDGFRα, KDR, or CD31 in staining media (5% FBS in HBSS) on ice for 30 minutes. After washing with staining media, cells were incubated with mouse IgG for 10 minutes (blocking step) before the secondary antibody was added for an additional 30 minutes. Cells were washed and re-suspended in staining media containing 1:3000 diluted Propidium Iodide (PI) and 10 μM of ROCK-inhibitor. Forward scatter versus side scatter and forward scatter height versus forward scatter width were used to exclude debris and doublets. PI positive population representing dead cells was excluded.

Cell sorting and analyses were performed using FACS Aria (Becton Dickenson). Data were analyzed using FlowJo software (Treestar). Sorted cells were collected in the differentiation media and were plated in V-shaped ultra-low attachment 96-well plates at a density of 5000 cells per well in the presence of 50 ng/ml FGF8, 50 ng/ml Wnt11, and 10 [symbol] M Rho-associated kinase (Rock-inhibitor; Calbiochem). The next day, cell aggregates were transferred to 6-well plates.

Immunofluorescence. Dissociated cells were cultured on glass cover slips for 2 days, fixed in 4% paraformaldehyde (Electron Microscopy Scientific) for 15 minutes, permeabilized when needed with 0.1% Triton x-100 (Fisher Scientific) in PBS for 5 minutes, and then blocked with 1% goat serum for 15 minutes. Cells were incubated for 2 hours with primary antibody at 37° C., washed three times and then incubated with a secondary antibody for an additional 1 hour. To induce the formation of tube like-structures, freshly sorted QP cells were cultured on Matrigel coated plates in the presence of StemPro34 containing 40 ng/ml of VEGF. Uptake of Ac-LDL by was assessed as previously described. All primary and secondary antibodies, catalog numbers, and vendors are listed in Table 1.

RT-PCR. For gene expression studies, RNA was purified using TRIzol reagent (Invitrogen) and complementary DNA was synthesized using Superscript III first strand cDNA synthesis kit according to manufacturer's protocol. Transcripts were amplified/detected for genes of interest using Taqman probes (listed in Table 2) and real-time PCR analysis was performed using an ABI Prism 7900HT (Applied Biosystems). Gene expression levels were estimated using the ΔΔCt method. Expression levels are all described in comparison to bulk, un-sorted populations.

Human fetal heart immunohistochemistry. De-identified human fetal hearts at different gestational ages were obtained from authorized sources and fixed in 4% PFA at 4° C. overnight. The hearts were washed three times with PBS and embedded in OCT after going through a sucrose gradient. Six-micrometer sections were stained with ROR2, CD13, KDR, PDGFRα, and CD31.

Patch clamp and field potential recording. Whole-cell patch-clamp recordings were performed using an EPC-10 patch-clamp amplifier (HEKA). The glass pipettes were prepared using borosilicate glass (Sutter Instrument, BF150-110-10) using a micropipette puller (Sutter Instrument, Model P-87). Current-clamp recording was conducted in normal Tyrode solution containing 140 mM NaCl, 5.4 mM KCl, 1 mM MgCl₂, 10 mM glucose, 1.8 mM CaCl₂, and 10 mM HEPES (pH 7.4 with NaOH at 25° C.) using the pipette solution: 120 mM K D-gluconate, 25 mM KCl, 4 mM MgATP, 2 mM NaGTP, 4 mM Na²-phospho-creatin, 10 mM EGTA, 1 mM CaCl₂ and 10 mM HEPES (pH 7.4 with KCl at 25° C.). Data were analyzed using Patchmaster and Igor Pro software. To characterize the electrophysiological properties of the QP population, cell aggregates were plated on fibronectin-coated MEA chambers in StemPro34 for 1-7 days. Spontaneous extracellular electrical activity was simultaneously recorded from 32 channels. Alternatively, external stimulation in the form of biphasic, anodic-first square pulses at a 10 ms duration was applied at a frequency of 1 Hz. The amplitude of the stimulation pulses ranged from 10, 30, and 60 mA. The local activation times calculated from analyzing the action potential morphology were then used to generate color-coded activation map to study conduction. All data was acquired by a custom-designed visualization and extraction tool written in Matlab TM.

In vivo analyses of QP cells. Mouse LAD ligation and cell injection. 5-10×10⁵ freshly sorted QP or QN cells derived from constitutive EGFP-hESC lines were re-suspended in 30 μl of differentiation media (containing no cytokines). The cell suspension (or media as control), were injected directly into the left ventricular (LV) wall of healthy NSG mice in an open chest procedure. Alternatively, the proximal left anterior descending artery of NSG mice was ligated to induce a sizable LV infarct, after which cell suspension, or media as control, was directly injected into the peri-infarct region. Hearts were harvested 8 weeks later, fixed in 4% PFA, and embedded in OCT after going through a sucrose gradient.

Heterotopic transplantation and cell injection. De-identified first trimester human hearts (gestational age 7-11 weeks) were obtained from authorized sources, immediately flushed with heparin solution (1:1000) and preserved in a modified University of Wisconsin Solution to minimize ischemic time during transfer (total time from harvest to transplantation<45 minutes). In the first model of ear-heart graft, the left ventricle was dissected from the human fetal heart and placed in the preservative solution. NSG mice were anesthetized with Avertin (37.5 mg/kg body weight) administered intraperitoneally and placed on a 37° C. warming mat. Using a finepointed scissor, a small incision was made at the base of the recipient ear caudal to the centrally located blood vessels. The incision was spread open and a pouch was created to deposit the human left ventricle at the distal end of the tunnel. Any air or residual fluid was gently expressed from the tunnel with light pressure and was sealed by gentle compression for 10-15 seconds without the use of sutures or adhesive bonding.

In the second model, late first trimester human fetal hearts were transplanted into the abdomen of NSG mice or nude rats to create a working heart model. The mice (or rats) were anesthetized with isoflurane (2%) and ketamine (25 mg/kg), and ventilated after oral intubation. An end-to-end anastomosis of the left atrium to the pulmonary artery, and ligation of the pulmonary veins and inferior vena cava, was performed on the human fetal hearts. After a left lateral thoracotomy, the human aorta was anastomosed to the murine abdominal aorta and the human superior vena cava was anastomosed to the murine inferior vena cava. After 7-10 days the abdominal wall was opened, exposing the beating human heart and 5×10⁵ constitutive EGFP-hESC-derived QP or QN cells were directly injected into the left ventricle.

For the ear-heart graft, cells were injected subcutaneously into the visible beating section of the grafted ventricular tissue. Hearts were harvested 8 weeks later, fixed in 4% PFA, and embedded in OCT after going through a sucrose gradient. The frozen tissue sections were obtained at 6 [symbol] m (for epifluorescence microscopy) or 30 pm (for confocal microscopy) and prepared for immunofluorescence staining using antibodies for GFP, Troponin, α-actinin, β-myosin heavy chain, CD31, vWF, and smooth muscle actin. In case of QN cell injection, visible teratoma formation was noted and the hearts were fixed in 4% PFA overnight followed by processing in 70% alcohol for an additional 24 h. The tissues were then embedded into a paraffin block for storage. All animal procedures were performed in accordance with Stanford Administrative Panel for Laboratory Animal Care guidelines.

Ca⁺⁺ Imaging. Human fetal hearts were explanted from the rat's abdomen and immediately sectioned at 200 pm thickness using a microtome. Next, tissue was placed in room temperature Tyrode's solution with 2.5 mM calcium chloride (2.5 mM CaCl, 0.1 g/L MgCl₂.6H₂O, 0.2 g/L KCl, 8.0 g/L NaCl, 1.0 g/L D-glucose, 4.0 g/L polyvinylpyrrolidone). Fluo-4 calcium dye was then added and videos were taken for 10 seconds of tissue electrically paced with a biphasic electrical stimulus having a pulse width of 10 ms, a pulse amplitude of 10 V peak-to-peak, and a pulse frequency of 1 or 2 Hz. Regions of interest were analyzed for dye fluorescence intensity changes, which were expressed as intensity ration f/f0 of each frame, with the resting fluorescence value f0 determined at the first frame of each video. Immediately after calcium dye imaging, hearts were stained with anti-GFP antibody and fluorescent images were taken to identify GFP-positive regions.

Data analysis. Data are shown as mean±standard deviation. Statistical analysis was performed with one-way analysis of variance (ANOVA) and nonparametric.

TABLE 1

| Antibody | Catalog no. | Vendor |
|---|---|---|
| ROR2 | MAB2064 | R&D Systems |
| ROR2 | 4105 | Cell Signaling |
| | | Technology |
| CD 13 | 301710 | Biolegend |
| KDR | MAB3572 | R&D Systems |
| KDR | ab62697 | Abcam |
| PDGFRα | 624008 | BD Biosciences |
| PDGFRα | ab61219 | Abcam |
| Nkx2.5 | ab97355 | Abcam |
| Nkx2.5 | AF2444 | R&D Systems |
| Tbx5 | Sc-17865 | Santa Cruz |
| MEF2C | 10056-1-AP | Proteintech |
| GATA4 | BAF2606 | R&D Systems |
| Troponin | MAB1691 | Millipore |
| Troponin | Sc-8118 | Santa Cruz |
| β Myosin Heavy Chain | Clone A4.951 | American Type |
| | | Culture |
| | | Collection |
| α-Actinin | A7811 | Sigma |
| Connexin 43 | MAB3068 | Millipore |
| Connexin 43 | AB1727 | Millipore |
| CD 31 | ab24590 | Abcam |
| CD 31 | 303118 | Biolegend |
| Von Willebrand Factor | ab6994-100 | Abcam |
| VE-Cadherin | 12-1449-82 | eBioscience |
| Smooth Muscle Actin | A2547 | Sigma |
| Smooth Muscle Myosin | BT-562 | Biomedical |
| | | Technologies |
| Anti-GFP | A21312 | Invitrogen |
| Streptavidin Qdot-605 | Q10101MP | Invitrogen |
| Goat anti-mouse Alexa fluor 594 | A11032 | Invitrogen |
| Donkey anti-mouse Alexa fluor 594 | A21203 | Invitrogen |
| Donkey anti-goat Alexa fluor 594 | A11058 | Invitrogen |
| Donkey anti-rabbit Alexa fluor 488 | A21206 | Invitrogen |
| Goat anti-rabbit Alexa fluor 488 | A11008 | Invitrogen |

TABLE 2

| Gene symbol | Assay ID |
|---|---|
| GAPDH | Hs99999905_m1 |
| OCT3/4 | Hs00742896_s1 |
| T (Brachyury) | Hs00610080_m1 |
| GOOSCOID | Hs00418279_m1 |
| MIXL1 | Hs00430824_g1 |
| MESP1 | Hs00251489_m1 |
| FOXA2 | Hs00232764_m1 |
| SOX17 | Hs00751752_s1 |
| KDR | Hs00911700_m1 |
| PDGFRα | Hs00998018_m1 |
| GATA4 | Hs00171403_m1 |
| MEF2C | Hs00231149_m1 |
| NKX2.5 | Hs00231763_m1 |
| TBX5 | Hs01052563_m1 |
| ISL1 | Hs01099687_m1 |
| CTNT | Hs00165960_m1 |
| Von Willebrand Factor | Hs00169795_m1 |
| Myosin-11 (Smooth muscle myosin) | Hs00224610_m1 |

1. A method of enriching for mammalian cardiovascular progenitor cells from a sample, the method comprising:
contacting said sample with a binding agent specific for a lineage specific marker present on said cardiovascular progenitor cells;
selecting for cells in the sample having binding agents bound thereto.

2. The method of claim 1, wherein the lineage specific marker is one or more of KDR, ROR2, CD13 and PDGFRa.

3. The method of claim 2, wherein the cardiovascular progenitor cells are selected for expression of KDR, ROR2, CD13 and PDGFRa.

4. The method according to claim 1, wherein said binding agent specific for a lineage specific marker is an antibody.

5. The method according to claim 1, wherein said selecting is performed by flow cytometry.

6. The method of claim 1, wherein said cardiovascular progenitor cells are human cells.

7. The method of claim 1 wherein sample comprises cardiovascular progenitor cells differentiated in culture from pluripotent cells.

8. The method of claim 7, wherein said pluripotent cells are ES cells or iPS cells.

9. The method of claim 7, wherein said culture comprises one or more of Bmp 4 and activin A.

10. The method of claim 9, wherein said culture initially comprises a wnt agonist, which is replaced with a wnt antagonist.

11. An enriched cell population obtained by the method set forth in claim 1.

12. The enriched cell composition according to claim 11, wherein at least about 50% of the total cells in said enriched cell population are cardiovascular progenitor cells.

13. The enriched cell composition according to claim 11, further comprising a physiologically acceptable excipient.

14. A method of providing cardiomyocytes to an individual in need thereof, the method comprising:
contacting a cell sample comprising cardiomyocyte progenitor cells with a binding agent specific for at least one lineage specific marker selected from KDR, ROR2, CD13 and PDGFRa;
selecting for cells in the sample having said binding agents bound thereto to provide for a cell population enriched in cardiomyocyte progenitors; and
introducing said cell population into said individual.

15. The method of claim 14, wherein the cardiovascular progenitor cells are selected for expression of KDR, ROR2, CD13 and PDGFRa.

16. The method of claim 15, wherein said cardiovascular progenitor cells are human cells.

17. The method of claim 1 wherein sample comprises cardiovascular progenitor cells differentiated in culture from pluripotent cells.
